(19) United States
(12) Patent Application Publication
(10) Pub. No.: US 2009/0325227 A1
(43) Pub. Date: Dec. 31, 2009

(54) C-TERMINUS MODIFICATION METHOD, C-TERMINUS IMMOBILIZATION METHOD AND ANALYSIS METHOD FOR PROTEIN OR PEPTIDE

(75) Inventors: Takashi Nakazawa, Nara (JP); Mutsumi Oka, Kyoto (JP); Kimiko Nishida, Osaka (JP); Minoru Yamaguchi, Kyoto (JP); Hiroki Kuyama, Kyoto (JP); Eiji Ando, Kyoto (JP); Norikazu Ueyama, Osaka (JP); Takaaki Okamura, Osaka (JP); Susumu Tsunasawa, Kyoto (JP)

Correspondence Address: Cheng Law Group, PLLC, 1100 17th Street, N.W., Suite 503, Washington, DC, 20036, US

(73) Assignees: SHIMADZU CORPORATION, Kyoto-shi, Kyoto (JP); NARA WOMEN'S UNIVERSITY, Nara-shi, Nara (JP); OSAKA UNIVERSITY, Suita-shi, Osaka (JP)

(21) Appl. No.: 12/090,747

(22) PCT Filed: Oct. 23, 2006
(86) PCT No.: PCT/JP2006/321583

§ 371 Date: Apr. 18, 2008

(30) Foreign Application Priority Data
Oct. 21, 2005 (JP) 2005-307831

(51) Int. Cl. C12P 21/00; C07K 1/107; G01N 33/68

(52) U.S. Cl. 435/69.1; 530/345; 436/86

(57) ABSTRACT

The present invention provides a method for inexpensively, easily and efficiently modifying the C-terminus of a protein or peptide; a method for easily and reliably isolating a C-terminal peptide fragment of a protein or peptide; and a method for rapidly, accurately and reliably determining an amino acid sequence of a protein or peptide by using a mass spectroscope. A method comprising the step of adding a formylation reagent and a catalyst to a protein or peptide to convert a carboxyl group into an aldehyde group. A method comprising the step of reacting a nucleophilic reagent with the aldehyde group to modify the C-terminus of the protein or peptide. A method comprising the step of reacting a support having a nucleophilic group to immobilize the protein or peptide. A method comprising the step of fragmenting the immobilized protein or peptide, washing the support, and isolating the C-terminal peptide fragment from the support. A method comprising the step of subjecting the isolated C-terminal peptide fragment to mass spectrometry and determining an amino acid sequence.

### TECHNICAL FIELD

The present invention relates to the field of protein/peptide chemistry. The present invention relates in particular to a method for modifying the C-terminus of a protein or peptide, a method for immobilizing the C-terminus of a protein or peptide, and a method for analyzing a protein or peptide. The method for modifying the C-terminus of a protein or peptide relates to a method for activating the C-terminus of a protein or peptide. The method for immobilizing the C-terminus of a protein or peptide relates to a method for trapping the C-terminus of a protein or peptide. The method for analyzing a protein or peptide relates to a method for determining an amino acid sequence of a protein or peptide.

### BACKGROUND ART

As a conventional method for modifying the C-terminus of a protein or peptide, the following method is conducted. For example, there is carried out a method wherein a condensation agent such as 1-ethyl-3-(3-dimethylaminopropyl)-carbodiimide hydrochloride (EDC) or an active ester such as N-hydroxysuccinimide (NHS) is used to activate the C-terminus of a protein or peptide and the activated C-terminus is reacted with a compound having a nucleophilic group such as an amino group, a hydroxyl group or a thiol group.

In the method using such a reagent, however, aspartic acid residues and glutamic acid residues of a protein or peptide are also activated for the nucleophilic reagent, thus making selective modification of the C-terminus infeasible in principle. Further, an unspecific intramolecular or intermolecular reaction also occurs between the reagent and the above nucleophilic functional groups present in side chains of a protein or peptide, so there is also unrealistic necessity for preceding protection of all side-chain functional groups of the above amino acids.

As virtually the sole method for chemically distinguishing the C-terminal carboxyl group of a protein or peptide from side-chain carboxyl groups of aspartic acid residues and glutamic acid residues, there is known a reaction for activating, as oxazolone, the C-terminal carboxyl group of the protein or peptide. An example of application of this reaction to peptide synthesis has been reported by Laplawy, M. T., Jones, D. S., Kenner, G. W., and Sheppard, R. C., in Tetrahedron, Vol. 11, pages 39-51 (1960).

The Dakin-West reaction for generating α-acylaminoketone via oxazolone in the presence of a base when an α-carboxyl group of an amino acid is treated with acetic anhydride has been reported by Dakin, H. D. West, R., in J. Biol. Chem. Vol. 78, pages 91-104, 745-756 (1928).

In Japanese Patent Application Laid-Open No. 10-90226, a peptide fragment obtained by previously chemically modifying the N- or C-terminus of a peptide so as to easily generate a product ion is simplified, and the peptide fragment containing the modified terminus is detected highly sensitively in analysis by MS/MS, and an amino acid sequence for the peptide is determined directly from a difference in the molecular weight of the peptide fragment.

In Japanese Patent Application Laid-Open No. 2005-139163, an oxazolone ring is formed in the C-terminus of a protein or peptide and then opened with a nucleophilic reagent to modify the C-terminus. An embodiment wherein a C-terminus-modified protein or peptide is obtained directly from the oxazoline ring, and a more efficient embodiment wherein the oxazolone ring is converted once into an active ester followed by yielding a C-terminus-modified protein or peptide, are disclosed.

Non-patent Document 1: Laplawy, M. T., Jones, D. S., Kenner, G. W., and Sheppard, R. C., in Tetrahedron, Vol. 11, pages 39-51 (1960)

Non-patent Document 2: Dakin, H. D. West, R., in J. Biol. Chem. Vol. 78, pages 91-104, 745-756 (1928)

Patent Document 1: Japanese Patent Application Laid-Open No. 10-90226

Patent Document 2: Japanese Patent Application Laid-Open No. 2005-139163

### DISCLOSURE OF THE INVENTION

### Object of the Invention

In the conventional activation/modification of the C-terminus of a protein or peptide via oxazolone, there are following problems. For example, there is a problem of a reduction in reaction efficiency due to hydrolysis of an activated carboxyl group. Particularly, this method is disadvantageous to selective modification of the C-terminus of a protein or peptide present at low concentration in an aqueous solution. There are also problems of poor solubility of a protein or peptide in an organic solvent suitable for preventing hydrolysis, and occurrence of many side reactions.

The modification/activation of the C-terminus of a protein or peptide by converting the C-terminus into an active ester can still not sufficiently overcome the problem of a reduction in reaction efficiency due to hydrolysis in an aqueous solution.

When the modification of the C-terminus via an active ester is carried out in this way, a modified group is introduced via an amide (peptide) bond or an ester bond into the C-terminus. In order to obtain a C-terminal peptide fragment from the C-terminus-modified protein or peptide thus obtained, the C-terminus-modified protein or peptide undergoes the action of a protease. However, when the C-terminal amino acid residue agrees in substrate specificity of the protease, the amide (peptide) bond or ester bond generated by the modification is hydrolyzed with the protease. That is, there is a serious problem that the effect of the modification is lost.

Accordingly, an object of the present invention is to provide a method for inexpensively, easily and efficiently modifying the C-terminus of a protein or peptide. Another object of the present invention is to provide a method for easily and reliably isolating a C-terminal peptide fragment of a protein or peptide. Still another object of the present invention is to provide a method for rapidly, accurately and reliably determining an amino acid sequence of a protein or peptide by using a mass spectroscope.

### SUMMARY OF INVENTION

The present inventors made extensive study, and as a result, they newly found that when a protein or peptide is treated in a system containing a formic acid and an acid anhydride, the oxazolone ring-forming reaction is accompanied by decarboxylation of the C-terminal carboxyl group of the protein or peptide, thereby introducing an aldehyde group derived from the formic acid, thus replacing the carboxyl group by the aldehyde group. Using this reaction, the introduced aldehyde group is modified, thereby achieving the object, and the present invention was completed.

The present invention encompasses the following inventions:

1. The following (1) to (3) relate to a method for converting the C-terminal carboxyl group of a protein or peptide into an aldehyde group.
(1) A method for converting a C-terminal carboxyl group of a protein or peptide into an aldehyde group, comprising:

adding, to a protein or peptide whose C-terminal carboxyl group is to be converted into an aldehyde group,

a formylation reagent selected from a mixture of a formic acid and an acid anhydride, a mixture of a formic acid and an acid halide, and an acid anhydride having a formyl group, and

a catalyst selected from a base catalyst, an acid catalyst and a phenol catalyst,

thereby converting the carboxyl group into an aldehyde group.

(2) The method according to the above-mentioned (1), wherein the acid anhydride in the mixture of a formic acid and an acid anhydride is selected from acetic anhydride, trifluoroacetic anhydride, benzoic anhydride, o-sulfobenzoic anhydride, propionic anhydride, and pentafluoropropionic anhydride.

The acid halide may be selected from formic acid chloride, acetyl chloride, acetyl bromide, and benzoyl chloride.

(3) The method according to the above-mentioned (1) or (2), wherein the acid anhydride having a formyl group is selected from a mixed anhydride of a formic acid and an acid other than a formic acid, and formic anhydride.
2. The following (4) to (11) relate to a method for modifying the C-terminus of a protein or peptide.
(4) A method for modifying the C-terminus of a protein or peptide, comprising the steps of:

converting the C-terminal carboxyl group of a protein or peptide whose C-terminus is to be modified, into an aldehyde group by the method of any one of the above-mentioned (1) to (3), and

reacting a nucleophilic reagent having an aldehyde-reactive group, with the aldehyde group, thereby modifying the C-terminus.

(5) The method according to the above-mentioned (4), wherein the nucleophilic reagent is reacted in formic acid.
(6) The method according to the above-mentioned (4) or (5),

wherein the aldehyde-reactive group is selected from an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—), a cysteinyl group (—COCH(NH₂)CH₂SH), and an active methylene group.

The reactive group is reacted with aldehyde thereby directly forming a double bond or a ring structure with carbon derived from the aldehyde. Such a double bond or a ring structure, unlike a peptide bond or an ester bond, does not undergo the action of general proteases.

The above double bond is formed with aldehyde-derived carbon and includes a C═N double bond. A structure containing such C═N double bond is preferably hydrazone, oxime, semicarbazone or the like.

The above ring structure is formed with aldehyde-derived carbon as one of ring constituent elements, and includes stable 5- or 6-membered rings not undergoing the action of general proteases. Such ring structure is preferably a thiazoline ring or the like.

(7) The method according to any one of the above-mentioned (4) to (6), wherein the aldehyde-reactive group in the nucleophilic reagent is substituted on, or bound to, an aromatic ring, an imidazoline ring and/or a fluorescent chromophore.
(8) The method according to any one of the above-mentioned (4) to (7), wherein the aldehyde-reactive group in the nucleophilic reagent is substituted on, or bound to, a charged group.
(9) The method according to the above-mentioned (8), wherein the charged group is involved in a charged amino acid residue.
(10) The method according to any one of the above-mentioned (4) to (9), wherein the nucleophilic reagent is selected from catechol, o-aminophenol, o-aminothiophenol, hydrazinobenzene, hydrazinopyrimidine, hydrazinoimidazoline, cysteine, 5-pyrazolone, and derivatives thereof.
(11) The method according to any one of the above-mentioned (4) to (10), wherein the nucleophilic reagent is selected from 3,4-dihyroxybenzoic acid, 2-hydrazino-4-trifluoromethyl pyrimidine, 2-hydrazino-2-imidazoline, 2-hydrazino-2-imidazoline hydrobromide salt, cysteinyl arginine, 1-methyl-3-phenyl-5-pyrazolone, and 3-methyl-1-phenyl-5-pyrazolone.
3. The following (12) to (19) relate to a method for immobilizing a protein or peptide via the C-terminus thereof.
(12) A method for immobilizing a protein or peptide onto a support, comprising the steps of:

converting the C-terminal carboxyl group of a protein or peptide whose C-terminus is to be immobilized, into an aldehyde group by the method of any one of the above-mentioned (1) to (3), and

reacting a support having an aldehyde-reactive group with the aldehyde group, thereby immobilizing the protein or peptide via the C-terminus thereof onto the support.

(13) The method according to the above-mentioned (12), wherein the support having an aldehyde-reactive group is reacted in formic acid.
(14) The method according to the above-mentioned (12) or (13), wherein the aldehyde-reactive group is selected from an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—), a cysteinyl group (—COCH(NH₂)CH₂SH), and an active methylene group.
(15) The method according to any one of the above-mentioned (12) to (14), wherein the aldehyde-reactive group is bound to the support via an aromatic ring, an imidazoline ring and/or a fluorescent chromophore.
(16) The method according to any one of the above-mentioned (12) to (15), wherein the aldehyde-reactive group is bound via a charged group to the support.
(17) The method according to the above-mentioned (16), wherein the charged group is involved in a charged amino acid residue.
(18) The method according to any one of the above-mentioned (12) to (17), wherein the support having an aldehyde-reactive group comprises a substance selected from catechol, o-aminophenol, o-aminothiophenol, hydrazinobenzene, hydrazinopyrimidine, hydrazinoimidazoline, cysteine, 5-pyrazolone, and derivatives thereof immobilized on the support.
(19) The method according to any one of the above-mentioned (12) to (18), wherein the support having an aldehyde-reactive group comprises a substance selected from 3,4-dihyroxybenzoic acid, 2-hydrazino-4-trifluoromethyl pyrimidine, 2-hydrazino-2-imidazoline, 2-hydrazino-2-imidazoline hydrobromide salt, cysteinyl arginine, 1-methyl-3-phenyl-5-pyrazolone, and 3-methyl-1-phenyl-5-pyrazolone immobilized on the support.
4. The following (20) and (21) relate to a method for selectively isolating a C-terminal peptide fragment of a protein or peptide.
(20) A method for isolating a C-terminal peptide fragment of a protein or peptide, comprising the step of:

immobilizing a C-terminus of a protein or peptide whose C-terminal peptide fragment is to be isolated, using a support having an aldehyde-reactive group by the method of any one of the above-mentioned (12) to (19),

fragmenting the protein or peptide with a protease to give a C-terminal peptide fragment immobilized on the support, and other non-immobilized peptide fragments,

washing the support to remove the other non-immobilized peptide fragments, and

releasing the immobilized C-terminal peptide fragment from the support to isolate the C-terminal peptide fragment.

(21) The method according to the above-mentioned (20), wherein when the support having an aldehyde-reactive group has a cysteinyl group as the reactive group and the cysteinyl group is immobilized via a charged group onto the support, the C-terminal peptide fragment is isolated so as to contain the charged group in the step of isolating the C-terminal peptide fragment.
5. The following (22) to (24) relate to a method for analyzing a protein or peptide.
(22) A method for analyzing a protein or peptide, comprising the steps of:

modifying, by the method of any one of the above-mentioned (4) to (11), the C-terminus of a protein or peptide to be analyzed, and

subjecting the modified protein or peptide to mass spectrometry.

(23) A method for analyzing a protein or peptide, comprising the steps of:

modifying, by the method of any one of the above-mentioned (4) to (11), the C-terminus of a protein or peptide to be analyzed,

fragmenting the modified protein or peptide to give a C-terminal peptide fragment having the modified C-terminus, and other peptide fragments, and

subjecting the C-terminal peptide fragment and the other peptide fragments to mass spectrometry.

(24) A method for analyzing a protein or peptide, comprising the steps of:

isolating, by the method of the above-mentioned (20) or (21), the C-terminus of a protein or peptide to be analyzed, and

subjecting the isolated C-terminal peptide fragment to mass spectrometry.

The present invention can provide a method for inexpensively, easily and efficiently modifying the C-terminus of a protein or peptide by converting the C-terminal carboxyl group of the protein or peptide selectively into an aldehyde group. In the modification method of the present invention, the C-terminal carboxyl group of a protein or peptide is converted selectively into an aldehyde group to enable the C-terminal carboxyl group to be modified while completely distinguishing it from other carboxyl groups present in side chains of protein or peptide. The aldehyde group, as compared with a C-terminal active group of oxazolone or active ester type, is free from reduction in reaction efficiency by hydrolysis in an aqueous solution, and is extremely easily distinguished from other functional groups in the protein or peptide, and thus there is an important advantage in that the C-terminus can be modified selectively without pretreatment such as protection of side chains of a protein or peptide in a biological sample.

Further according to the present invention, the resulting C-terminal modified protein has a modified group introduced thereinto via a bond (for example, a C═N double bond-containing hydrazone, oxime, semicarbazone, thiazolidine ring, etc.) other than a peptide bond or an ester bond, thus not undergoing the action of general proteases, so whichever protease is used, the bond can be maintained in the process for obtaining the C-terminal peptide fragment, thereby ensuring the effect of the modification. The present invention, therefore, can provide a method for easily and accurately isolating a C-terminal peptide fragment of a protein or peptide. The present invention can also provide a method for rapidly, accurately and reliably determining an amino acid sequence of a protein or peptide by using a mass spectroscope. The C-terminal peptide fragment isolated by the present invention is subjected to determination of an amino acid sequence from the C-terminus by mass spectrometry, thereby making highly reliable proteome analysis possible. There is also an important advantage that the amino acid sequence determination method of the present invention can be combined with known methods for determining an N-terminal amino acid sequence of a protein or peptide to make more highly reliable proteome analysis possible.

### BRIEF DESCRIPTION OF THE DRAWINGS

FIG. 1 shows the results of mass analysis of digests of a modified protein obtained by modifying a protein sample (soybean trypsin inhibitor) with a formic acid/trifluoroacetic anhydride mixture as a formylation reagent and with 2-hydrazino-2-imidazoline hydrobromide salt as a nucleophilic reagent in Example 1.

FIG. 2 shows the results of mass analysis of digests of a modified protein obtained by modifying a protein sample (soybean trypsin inhibitor) with a formic acid/acetic anhydride mixture as a formylation reagent and with 2-hydrazino-2-imidazoline hydrobromide salt as a nucleophilic reagent in Example 2.

FIG. 3 shows the results of mass analysis of digests of a modified protein obtained by modifying a protein sample (horse cytochrome c) with a formic acid/trifluoroacetic anhydride mixture as a formylation reagent and with 2-hydrazino-2-imidazoline hydrobromide salt as a nucleophilic reagent in Example 3.

FIG. 4 shows the results of mass analysis of a C-terminal peptide fragment isolated from digests obtained by digesting a modified protein that is a protein sample (horse cytochrome c modified with a formic acid/trifluoroacetic anhydride mixture as a formylation reagent and with a cysteinyl arginine-immobilized resin as a nucleophilic agent in Example 4.

FIG. 5 shows the results of mass analysis of a peptide mixture obtained by modifying a peptide sample (leucine enkephalin) with a formic acid/acetic anhydride mixture as a formylation reagent and with 2-imidazolino-2-hydrazine hydrobromide salt as a nucleophilic reagent in Example 5.

### MODES FOR CARRYING OUT THE INVENTION

### 1. Conversion of the C-Terminal Carboxyl Group of a Protein or Peptide into an Aldehyde Group

In the present invention, the first method is a method for converting the C-terminal carboxyl group of a protein or peptide into an aldehyde group via selective activation. Specifically in this method, a formylation reagent is allowed to act on a protein or peptide whose C-terminus is to be converted into aldehyde, in the presence of a suitable catalyst, thereby yielding a protein or peptide whose C-terminus has been converted into an aldehyde group.

In the first method, the C-terminus of a protein or peptide is converted into an aldehyde group, probably via the mechanism shown in following scheme (I). Specifically, the carboxyl group is converted into an aldehyde group, probably via formation of an oxazolone ring in the C-terminus, formulation of the oxazolone ring and decarboxylation. The following scheme (I) shows an example wherein a mixture of formic acid and acetic anhydride is used as a formylation reagent. NHCHR¹CO, NHCHR²CO, . . . , NHCHRⁿ⁻¹CO, and NHCHRⁿCO mean an amino acid residue.

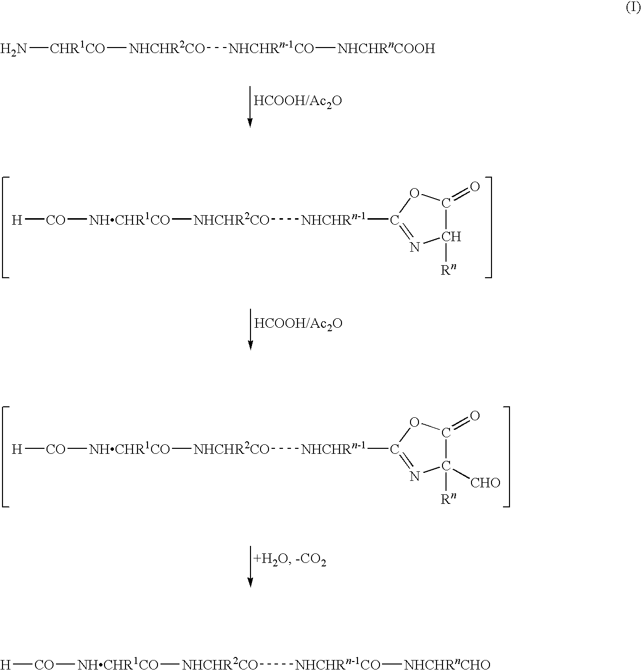

Herein, an oxazolone ring is not formed with a carboxylic group in a side chain of an aspartic acid residue or a glutamic acid residue. It follows that in the reaction of the invention estimated to proceed via formation of an oxazolone ring, the C-terminus of a protein or peptide can be selectively converted. That is, the conventionally required previous protection of side-chain carboxyl groups of a protein or peptide, and amino groups, hydroxyl groups, and the like which may react with the activated carboxyl group, is not necessary. Note that simultaneously with this oxazolone formation, the N-terminal amino group is formylated with the acid anhydride in formic acid, as shown in the scheme (I), and automatically protected from the reaction with the activated carboxyl group.

As the formylation reagent is used a reagent that realizes oxazolone-ring formation estimated to occur in the C-terminus of a protein or peptide and introduction of a formyl group into the oxazolone ring and that contains a source of supplying a formyl group to be introduced for formylating the oxazoline ring. Specifically, at least one is selected from a mixture of a formic acid and an acid anhydride (formic acid/acid anhydride mixture), a mixture of a formic acid and an acid halide (formic acid/acid halide mixture), and an acid anhydride having a formyl group.

When a formic acid/acid anhydride mixture or a formic acid/acid halide mixture is used, formic acid is reacted with the acid anhydride or acid halide, thereby generating an acid anhydride having a formyl group. The thus formed acid anhydride having a formyl group realizes formation of an oxazolone ring and introduction of a formyl group into the oxazolone ring.

In the formic acid/acid anhydride mixture, examples of the acid anhydride include, but are not limited to, acetic anhydride, trifluoroacetic anhydride, benzoic anhydride, o-sulfobenzoic anhydride, propionic anhydride, and pentafluoropropionic anhydride and the like. The acid anhydride is preferably an acid anhydride of a strongly acidic carboxylic acid having an electron-withdrawing group. Accordingly, the acid anhydride is preferably selected from trifluoroacetic anhydride and pentafluoropropionic anhydride among the above-mentioned acid anhydrides. One or more acid anhydrides may be selected therefrom.

In the formic acid/acid halide mixture, examples of the acid halide include, but are not limited to, formic acid chloride, acetyl chloride, acetyl bromide, and benzoyl chloride and the like. One or more acid halides may be selected therefrom.

When a formic acid/acid anhydride mixture and/or a formic acid/acid halide mixture is used as the formylation reagent, formic acid is preferably used in an equal or excess amount relative to an amount of an acid anhydride and/or an acid halide on a molar basis. This is because when an acid anhydride and/or an acid halide is present in an amount larger than an amount of formic acid, the acid anhydride and/or the acid halide may react directly with a protein or peptide. Specifically, a formic acid and an acid anhydride and/or an acid halide may be used in a mixing ratio of for example 1:1 to 4:1, preferably 2:1 on a molar basis. When a formic acid/acid anhydride mixture and/or a formic acid/acid halide mixture are/is used as the formylation reagent, an organic solvent other than formic acid may not be particularly used.

The formulation reagent is used in an excess amount relative to a protein or peptide. For example, the formylation reagent is used preferably in such a large excess amount that a protein or peptide is dissolved at a concentration of 1 mM or less in the formulation reagent. If the concentration of a protein or peptide in the reaction solution is higher than 1 mM, there may occur crosslinking based on formation of an amide bond or an ester bond between protein or peptide molecules.

On the other hand, the acid anhydride having a formyl group is an acid anhydride derived from at least formic acid, and includes a mixed anhydride of a formic acid and an acid other than a formic acid, and a formic anhydride. The mixed anhydride of a formic acid and an acid other than a formic acid includes, for example, a mixed anhydride of formic acid and acetic acid. When an acid anhydride having a formyl group is used as the formylation reagent, it may be reacted in the absence of a solvent, may be reacted in the presence of formic acid as a solvent or may be reacted in the presence of an organic solvent other than formic acid.

The formylation reagent is used in an excess amount relative to a protein or peptide. For example, when the reaction is carried out in the absence of a solvent, the formylation reagent is used preferably in such a large excess amount that a protein or peptide is dissolved at a concentration of 1 mM or less in the formylation reagent. When the reaction is carried out in the presence of a solvent, the formylation reagent may be in an excess amount relative to an amount of a protein or peptide such that the protein or peptide can be dissolved at a concentration of 1 mM or less in a mixed solution of the formylation reagent and the solvent. If the concentration of a protein or peptide in the reaction solution is higher than 1 mM, there may occur crosslinking, in the same manner as described above, based on formation of an amide bond or an ester bond between protein or peptide molecules.

On the other hand, in withdrawal of C^{α}-hydrogen from the C-terminal amino acid residue after formation of an oxazolone ring and in subsequent formylation and decarboxylation reaction, a catalyst is necessary. As the catalyst, one or more catalysts are selected from a base catalyst, an acid catalyst and a phenol catalyst. The base catalyst includes typical bases such as triethylamine, N,N-dimethylaminopyridine (DMAP), etc., and one or more bases are selected therefrom. The phenol catalyst is estimated to act as a base catalyst in formic acid or in another substance containing a formyl group, but may also act as an acid catalyst depending on the case. Examples of the phenol catalyst include pentafluorophenol, p-nitrophenol, etc. In the present invention, relatively highly acidic phenols are also preferably used.

These catalysts may be used while being dissolved at a suitable concentration in a solvent or in a substance (e.g. formic acid) usable for the formylation reagent.

The catalyst, in the case of the phenol catalyst, may be used in 50- to 100-fold large excess amount relative to an amount of a protein or peptide on a molar basis, thereby giving good results. The amount of the catalyst other than the phenol may be suitably determined by those skilled in the art. Particularly, when the phenol catalyst is used in combination with a base catalyst, the base catalyst may be used in 1- to 10-fold amount relative to an amount of a protein or peptide on a molar basis. While the acid catalyst may not be particularly used when formic acid as the formylation reagent is used in an amount for use as a solvent, the acid catalyst may be used in 1- to 10-fold amount relative to an amount of a protein or peptide on a molar basis when a general organic solvent other than formic acid is used.

In order to convert the C-terminus of a protein or peptide into aldehyde, the reaction may be carried out for example under the conditions of 30 to 70° C. and 10 to 30 minutes.

An aldehyde group is easily oxidized in the air so that particularly when the amount of a protein or peptide aldehydated is low and the like, the reaction shall not be conducted for a long time, and a product shall not be left in the air. In order to prevent the generated aldehyde from contacting with the air, for example, it is preferable that the reaction is carried out in an atmosphere of an inert gas such as nitrogen or argon. Furthermore, preferably, the generated aldehyde is placed in the above inert gas atmosphere until the next reaction is conducted to be prevented from contacting with the air.

The method for converting the C-terminal carboxyl group of a protein or peptide into an aldehyde group may be effectively applied to second to fifth methods described below.

### 2. Method for Modifying the C-Terminus of a Protein or Peptide

In the present invention, the second method is a method for modifying the C-terminus of a protein or peptide. This method comprises converting the C-terminal carboxyl group of a protein or peptide whose C-terminus is to be modified, into an aldehyde group by the first method described above, and then allowing a nucleophilic reagent to act on the aldehyde group, thereby giving a protein or peptide whose C-terminus has been modified.

In the second method, the C-terminus is converted into an aldehyde group as shown in the above scheme (I), and then the nucleophilic addition reaction of a nucleophilic reagent to the aldehyde group occurs as shown in following scheme (II), thereby giving a protein or peptide whose C-terminus has been modified. The following scheme (II) shows an example wherein an amino group-containing compound represented by the general formula X-NH₂ is used as the nucleophilic reagent. NHCHR¹CO, NHCHR²CO, . . . , NHCHRⁿ⁻¹CO, NHCHRⁿCO mean an amino acid residue.

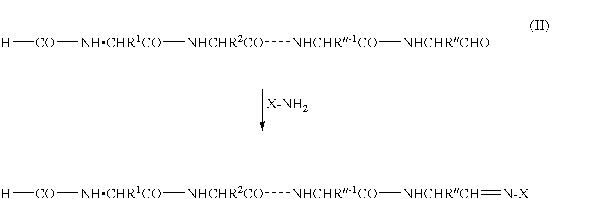

The nucleophilic reagent is a reagent having a group reactive to an aldehyde group. The aldehyde-reactive group may be selected from nucleophilic groups such as an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—), a cysteinyl group (—COCH(NH₂)CH₂SH), and an active methylene group. Preferably selected from the above-mentioned groups from the viewpoint of product stability are a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—) or a cysteinyl group (—COCH(NH₂)CH₂SH). One or more reactive groups may be selected therefrom. The active methylene group as used herein refers to a methylene group adjacent to a carbonyl group, a methylene group put between carbonyl groups, and a methylene group put between unsaturated bonds.

Particularly, a hydrazino group (—NHNH₂), a hydroxylamino group (—NHOH), a semicarbazido group (—NHCONHNH₂) or the like is preferable in that it can react with aldehyde to directly form a double bond with carbon derived from the aldehyde. The double bond as used herein is formed with carbon derived from the aldehyde, and includes a C═N double bond. A structure containing such C═N double bond includes preferably hydrazone, oxime, semicarbazone or the like. Such double bond, unlike a peptide bond or an ester bond, is not liable to the action of general proteases.

The amino group- and thiol group-substituted ethylene group (—CH(NH₂)—CH(SH)—), the amino group- and thiol group-substituted ethene group (—C(NH₂)═C(SH)—) and the cysteinyl group (—COCH(NH₂)CH₂SH) and the like are preferable in that they can react with aldehyde to directly form a ring structure with carbon derived from the aldehyde. The ring structure as used herein is formed with carbon derived from the aldehyde as one of ring constituent elements, and includes stable 5- or 6-membered rings not liable to the action of general proteases. Such ring structure is preferably a thiazoline ring or the like.

As the nucleophilic reagent, a reagent having the above reactive group bound to a suitable structure may be used. For example, the reactive group may be substituted on, or bound to, an aromatic ring, an imidazole ring or the like. The aromatic ring includes heterocycles such as a pyrimidine ring besides a benzene ring. The reactive group may be substituted on, or bound to, a fluorescent chromophore.

The fluorescent chromophore includes coumarin derivatives such as 7-aminocoumarin and 7-hydroxycoumarin, fluorescein, and fluorescein derivatives such as fluorescein amine. In this case, for example a protein or peptide modified by this method is fragmented by a suitable method, and the resulting peptide fragment mixture is separated with high performance liquid chromatography (HPLC) while monitoring with a fluorescence detector, whereby the C-terminal peptide fragment can be selectively and easily separated and purified.

The reactive group may be substituted on, or bound to, a charged group. The charged group is preferably a group carrying a stable positive charge or a stable negative charge, and the stable positive charge includes charges derived from a guanidino group, etc., and the stable negative charge includes charges derived from a sulfonyl group, etc. Specific examples of the charged group include amino acid residues carrying the positive or negative charges mentioned above.

Among them, as a hydrazine derivative having a hydrazino group as a reactive group, a hydroxylamine derivative having a hydroxylamino group, and a semicarbazido derivative having a semicarbazido group, those generally known as carbonyl reagents may be used without particular limitation. The hydrazine derivative, hydroxylamine derivative and semicarbazido derivative react with aldehyde groups to generate chemically stable hydrazone, oxime and semicarbazone, respectively.

Specifically, the nucleophilic reagent may be selected from catechol, o-aminophenol, o-aminothiophenol, hydrazinobenzene, hydrazinopyrimidine, hydrazinoimidazoline, cysteine, 5-pyrazolone, and derivatives thereof.

More specifically, the catechol derivative includes 3,4-dihyroxybenzoic acid, etc. The hydrazinopyrimidine derivative includes 2-hydrazino-4-trifluoromethyl pyrimidine, etc. The hydrazinoimidazoline derivative includes 2-hydrazino-2-imidazoline, 2-hydrazino-2-imidazoline hydrobromide salt, etc. The cysteine derivative includes cysteinyl arginine, etc. The 5-pyrazolone derivative includes 1-methyl-3-phenyl-5-pyrazolone, 3-methyl-1-phenyl-5-pyrazolone, etc.

Particularly, the embodiment in which the nucleophilic reagent has a charged group is effective in a fifth method (method for analyzing a protein or peptide) described later. That is, when the modified protein peptide is further fragmented to give a mixture of a C-terminal peptide fragment having the modified C-terminus and other peptide fragments, the mixture can be subjected directly to mass spectrometry without isolation of the C-terminal peptide fragment. Accordingly, analysis efficiency can be significantly increased (this will be described later in detail in the fifth method).

The nucleophilic reagent may be used in 1- to 100-fold amount relative to an amount of a protein or peptide on a molar basis. Particularly, good results can be obtained by using the nucleophilic reagent in 50- to 100-fold large excess amount. The nucleophilic reagent used has a relatively low molecular weight, so even if it is used in large excess amount, its influence can be suppressed by using a suitable measurement method in MALDI-MS. Fundamentally, the reaction will proceed when the nucleophilic reagent is used in 50-fold or less amount relative to an amount of a protein or peptide on a molar basis, for example in about 1- to 10-fold molar amount. However, the nucleophilic reagent is used desirably at a concentration as high as possible in order that the reaction time is reduced to 10 hours or less.

These nucleophilic reagents may be used while being dissolved for example in the following solvents.

The reaction between the nucleophilic reagent and the protein or peptide whose C-terminus has been converted into aldehyde may be carried out for example in a solvent selected from formic acid, water, and organic solvents such as dimethylformamide (DMF), dimethyl sulfoxide (DMSO) and N-methylpyrrolidone. When water is used as the solvent, urea, guanidine hydrochloride or a surfactant such as SDS may be allowed to be coexistent in order to solubilize the protein or peptide. Preferably, the concentration of the protein or peptide in the solvent is for example in the range of about 0.1 mM to 1 mM. However, the concentration of the protein or peptide in the solvent is not necessarily limited to this range because the concentration varies depending on the kind of the nucleophilic reagent.

The reaction between the nucleophilic reagent and the protein or peptide whose C-terminus has been converted into aldehyde will proceed generally under neutral conditions (in the vicinity of pH 7) to alkaline conditions (about pH 9) in an aqueous solution. When water is used as the solvent, adjustment of pH may be carried out using general bases such as sodium bicarbonate and sodium hydroxide. When the reaction is carried out in an organic solvent, bases such as pyridine, triethylamine, N,N-dimethylaminopyridine may be used. Preferably, this reaction is carried out for example under the conditions of 20 to 50° C. and 2 to 10 hours.

### 3. Method for Immobilizing the C-Terminus of a Protein or Peptide

In the present invention, the third method is a method for immobilizing a protein or peptide via its C-terminus. This method comprises converting the C-terminal carboxyl group of a protein or peptide whose C-terminus is to be immobilized, into an aldehyde group by the first method described above, and then allowing a support having an aldehyde-reactive group (also referred to hereinafter as a “nucleophilic agent”) to act as a nucleophilic agent thereon, thereby giving a protein or peptide whose C-terminus has been immobilized. That is, the third method is the same as the second method except that the nucleophilic agent, that is, a support having an aldehyde-reactive group, is used in place of the nucleophilic reagent.

The support is not particularly limited insofar as it is an insoluble solid support. Herein, the term “insoluble” means that the support is insoluble under the environment where it is placed (for example, the environment where a protein or peptide which has been converted to aldehyde is reacted with the nucleophilic agent). Specific examples of such supports include synthetic resins, glass beads, etc.

The aldehyde-reactive group is the same as described in the second method and may be selected from nucleophilic groups such as an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)CH(SH)—), an amino group- and thiol group-substituted ethene group (—C(NH₂)═C(SH)—), a cysteinyl group (—COCH(NH₂)CH₂SH), and an active methylene group. One or more reactive groups may be selected therefrom.

The nucleophilic agent is not particularly limited insofar as it is a support having the above reactive group. The reactive group may be bound to the support directly or indirectly via a suitable structure. For example, the reactive group may be bound to the support via an aromatic ring, an imidazoline ring or the like. The aromatic ring includes heterocycles such as a pyrimidine ring besides a benzene ring. The reactive group may be bound to the support via a fluorescent chromophore. The fluorescent chromophore includes coumarin derivatives such as 7-aminocoumarin and 7-hydroxycoumarin, fluorescein, and fluorescein derivatives such as fluorescein amine.

The reactive group may be bound to the support via a charged group. The charged group is preferably involved in a group carrying a stable positive charge or a stable negative charge, and the stable positive charge includes a guanidino group, etc., and the stable negative charge includes a sulfonyl group, etc. Specific examples of the charged group include amino acid residues carrying the positive or negative charges as described above. In these cases, the reactive group is substituted on, or bound to, the above intervening structure, and thereby bound to the support indirectly via the intervening structure.

Among them, when a support to which the hydrazine derivative having a hydrazino group as a reactive group, the hydroxylamine derivative having a hydroxylamino group or the semicarbazido derivative having a semicarbazido group has been bound is used as the nucleophilic agent, such derivatives include those known generally as carbonyl reagents. Herein, the hydrazine derivative, hydroxylamine derivative and semicarbazido derivative will react with aldehyde groups to form chemically stable hydrazone, oxime and semicarbazone, respectively.

Specific examples of the nucleophilic agent that may be used include supports on which a substance selected from catechol, o-aminophenol, o-aminothiophenol, hydrazinobenzene, hydrazinopyrimidine, hydrazinoimidazoline, cysteine, 5-pyrazolone, and derivatives thereof has been immobilized.

Specific examples of such nucleophilic agents that can be used include supports on which catechol derivatives such as 3,4-dihyroxybenzoic acid, hydrazinopyrimidine derivatives such as 2-hydrazino-4-trifluoromethyl pyrimidine, or hydrazinoimidazoline such as 2-hydrazino-2-imidazoline and 2-hydrazino-2-imidazoline hydrobromide salt have been immobilized. Supports on which cysteine derivatives such as cysteinyl arginine have been immobilized may also be used as the nucleophilic agent. Supports on which 5-pyrazolone derivatives such as 1-methyl-3-phenyl-5-pyrazolone and 3-methyl-1-phenyl-5-pyrazolone have been immobilized may also be used as the nucleophilic agent.

Particularly, embodiment in which the nucleophilic agent has a cysteinyl group as the aldehyde-reactive group is effective in a fourth method (method for isolating a C-terminal peptide fragment of a protein or peptide) described later. That is, when the immobilized protein peptide is further fragmented and a C-terminal peptide fragment having an immobilized C-terminus is isolated, the C-terminal peptide fragment with an intervening structure (that is, a structure intervening between the support and the reactive group in the nucleophilic agent) maintained in the C-terminal peptide fragment can be released from the support. Accordingly, this embodiment is very effective in analysis of the protein or peptide by utilizing the physical properties and the like of the intervening structure (this will be described in detail in the fourth method).

The embodiment in which the nucleophilic agent has a structure carrying a charge as an intervening structure is effective in a fifth method (method for analyzing a protein or peptide) described later. That is, the C-terminal peptide fragment with the intervening structure (that is, the charged structure) maintained therein is released as described above, thus facilitating generation of molecular ions and generation of fragment ions in mass spectrometry. Accordingly, the accuracy and reliability in analysis of the protein or peptide by mass spectrometry can be significantly increased (this will be described in detail in the fifth method).

The nucleophilic agent can be used so that its reactive group is in 1- to 100-fold amount relative to an amount of a protein or peptide on a molar ratio. Particularly, good results can be obtained by using in 50- to 100-fold large excess amount. Fundamentally, the reaction will proceed when the nucleophilic agent is used so that its reactive group is in 50-fold or less amount relative to an amount of a protein or peptide on a molar basis, for example in 1- to 10-fold molar amount. However, the nucleophilic agent is used desirably at a concentration as high as possible in order that the reaction time is reduced to 10 hours or less.

The reaction between the nucleophilic agent and the protein or peptide whose C-terminus has been converted into aldehyde may be carried out for example in a solvent selected from formic acid, water, and organic solvents such as dimethylformamide (DMF), dimethyl sulfoxide (DMSO) and N-methylpyrrolidone. When water is used as the solvent, urea, guanidine hydrochloride and a surfactant such as SDS may be allowed to be coexistent in order to solubilize the protein or peptide. Preferably, the concentration of the protein or peptide in the solvent is for example in the range of about 0.1 mM to 1 mM. However, the concentration of the protein or peptide in the solvent is not necessarily limited to this range because the concentration varies depending on the kind of the nucleophilic agent.

The reaction between the nucleophilic reagent and the protein or peptide whose C-terminus has been converted into aldehyde will proceed generally under neutral conditions (in the vicinity of pH 7) to alkaline conditions (about pH 9) in an aqueous solution. When water is used as the solvent, adjustment of pH may be carried out using general bases such as sodium bicarbonate and sodium hydroxide. When the reaction is carried out in an organic solvent, bases such as pyridine, triethylamine, N,N-dimethylaminopyridine may be used. Preferably, this reaction is carried out for example under the conditions of 20 to 50° C. and 2 to 10 hours.

### 4. Method for Isolating a C-Terminal Peptide Fragment of a Protein or Peptide

In the present invention, the fourth method is a method for isolating a C-terminal peptide fragment of a protein or peptide. This method comprises the steps of immobilizing a protein or peptide whose C-terminal peptide fragment is to be isolated, via its C-terminus onto a support by the third method described above, then fragmenting the immobilized protein or peptide, washing the support, and isolating the C-terminal peptide fragment. In this method, the C-terminal peptide fragment is isolated via the mechanism shown in following scheme (III). The following scheme (III) shows an example where a resin having a cysteinyl group bound to the resin via an arginine residue (that is, a resin having cysteinyl arginine immobilized thereon) is used as the nucleophilic agent. NHCHR¹CO, NHCHR²CO, . . . , NHCHRⁿ⁻¹CO, NHCHRⁿCO mean an amino acid residue. In the arginine residue, a guanidino group is protected as a sulfonamido group with a pentamethylhydrobenzofuran-5-sulfonyl (Pdf) group.

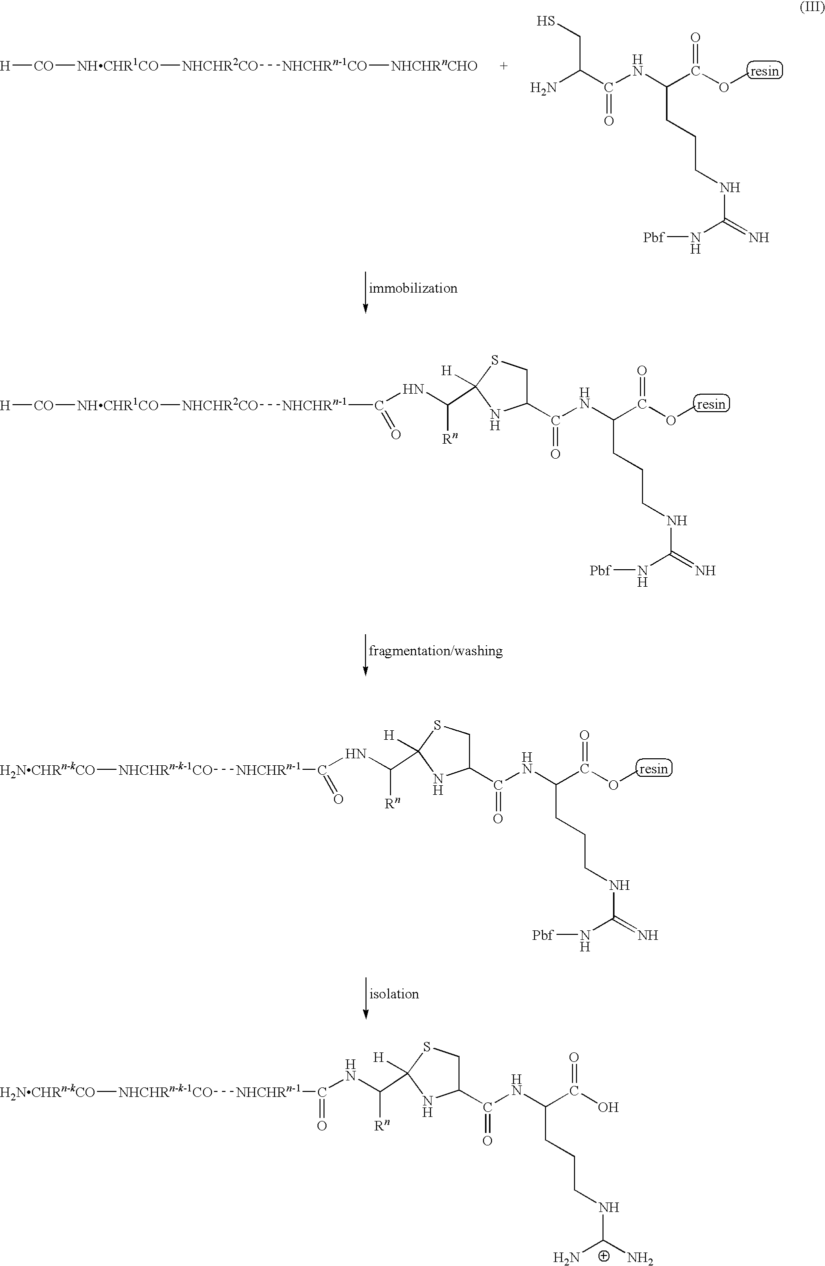

The immobilization step is carried out according to the third method described above. In the fourth method, the nucleophilic agent may be subjected to suitable protection in consideration of conditions in steps carried out after immobilization. For example, when the nucleophilic agent has a guanidino group in a part of the intervening structure thereof as shown in the example in the above scheme (III), the guanidino group may be protected with a protective group such as a pentamethylhydrobenzofuran-5-sulfonyl (Pdf) group, a 2,2,5,7,8-pentamethylchromane-6-sulfonyl (Pmc) group, a 2-mesitylenesulfonyl (Mts) group or the like. When a support for solid-phase peptide synthesis based on the Fmoc (fluorenylmethoxycarbonyl)method is used, a Pbf group, a Pmc group or the like is preferably used as a protective group suitable for the Fmoc method. When a support for solid-phase peptide synthesis based on the Boc (t-butoxycarbonyl)method is used, an Mts group or the like is preferably used as a protective group suitable for the Boc method.

Protection of the guanidino group can be expected to bring about an effect of preventing an ester bond between an arginine residue and the support from being decomposed upon a trypsin treatment in a fragmentation step described later. The C-terminal peptide fragment when subjected to strongly acidic conditions in a releasing step described later is released from the support and simultaneously the protective group is also removed.

In the immobilization step, particularly where a resin having cysteinyl arginine immobilized thereon is used, a thiazoline ring is formed by the reaction between the aldehyde group and the cysteinyl group as shown in the above scheme (III).

In the fragmentation step, the protein or peptide immobilized by the third method described above is fragmented, thereby giving the C-terminal peptide fragment immobilized on the support and other peptide fragments. The fragmentation method may be either a chemical method or a biochemical method as long as fragmentation is effected under the conditions where the bond between the support and the C-terminus is not cut. Preferably, fragmentation is carried out by biochemical methods using proteases having relatively high substrate specificity, such as chymotrypsin, trypsin, Glu-C (V8) protease, a lysyl endopeptidase and an Asp-N endopeptidase or by chemical methods using chemical reagents such as cyanogen bromide (BrCN). In this respect, a chemical structure derived from the C-terminal aldehyde group used in immobilization (that is, a double bond or a cyclic structure formed directly with carbon derived from the aldehyde) does not undergo the hydrolysis action of proteases. The double structure and cyclic structure are as previously described in the second method. Accordingly, enzymes that can be used in fragmentation can be selected more freely than those used after immobilization of the C-terminus via an amide bond or an ester bond. That is, the selectable range of usable methods can be made broader. Therefore, the possible fragmentation method is not limited to the above-illustrated methods. Specific conditions for fragmentation may be suitably determined by those skilled in the art, depending on reagents and enzymes used.

In a washing step, from the mixture of the C-terminal peptide fragment immobilized on the support and other peptide fragments, the other peptide fragments are removed by washing the support, while the C-terminal peptide fragment immobilized on the support is left. As a wash fluid for washing the support, it is enough to generally use a buffer solution used in enzymatic digestion as it is. When unnecessary peptide fragments may have been insolubilized, a suitable surfactant may be added to the buffer solution. It is however necessary to avoid washing with a buffer solution that is such extreme acidic or alkaline so as not to cause release of the protein or peptide immobilized on the support.

In an isolation step, for the C-terminal peptide fragment immobilized on the support, the C-terminal peptide fragment is released from the support by the chemical method. At this time, the C-terminal peptide fragment immobilized on the support is decomposed preferably at the site between the support and the intervening structure in the nucleophilic agent used in immobilization (that is, the structure intervening between the support and the reactive group in the nucleophilic agent) so that the released C-terminal peptide fragment can maintain the intervening structure therein. For this purpose, the nucleophilic agent may be designed so as to form a chemically stable structure by the reaction between the aldehyde group and the nucleophilic agent during immobilization. Releasing conditions not causing undesirable decomposition of the structure formed by the reaction between the aldehyde group and the nucleophilic agent during immobilization may be suitably determined by those skilled in the art.

Particularly as shown in the above scheme (III), a thiazoline ring is preferably formed in immobilization. The thiazoline ring is chemically more stable than, for example, hydrazone, oxime and semicarbazone that are formed by the reaction between aldehyde and hydrazine, hydroxylamine and semicarbazide, respectively. Accordingly, the thiazoline ring will not be decomposed even upon exposure for example to strongly acidic conditions during releasing the C-terminal peptide fragment from the support. That is, the C-terminal peptide fragment is cut off from the support such that the fragment can maintain the intervening structure therein, as shown in the above scheme (III).

A composition of a releasing fluid and reaction conditions for releasing vary depending on the reactive group bound to the support, etc., and may be suitably determined by those skilled in the art, depending on the kind of the nucleophilic agent used. For example, when the nucleophilic agent capable of forming a thiazoline ring as described above, that is, a support having an amino group- and thiol group-substituted ethylene group (—CH(NH₂)—CH(SH)—) or an amino group- and thiol group-substituted ethene group (—C(NH₂)═C(SH)—), or a support having cysteine, o-aminothiophenol or a derivative thereof bound thereto is used, a strongly acidic releasing fluid comprising a strong acid such as trifluoroacetic acid as a main component may be used. When such a releasing fluid is used, releasing may be effected under the conditions of room temperature and 3 to 5 hours, preferably about 4 hours.

To isolate the C-terminal peptide fragment so as to maintain the intervening structure therein is effective where the intervening structure is established for analysis of the protein or peptide. In this case, the protein or peptide can be analyzed by utilizing the physical properties and the like of the intervening structure. For example, when the intervening structure is a charged group, the protein or peptide can be effectively analyzed by mass spectrometry. This will be described in a fifth method described later.

When the chemical structure derived from the C-terminal aldehyde group used in immobilization is particularly a C═N double bond, the C═N double bond can be cleaved under chemical conditions completely different from those for cleaving an amide bond (peptide bond) constituting a peptide. Accordingly, the C-terminal peptide fragment immobilized on the support is released by cleavage of the C═N double bond, and then the resulting aldehyde group can be subjected again to other derivatization. Specific chemical conditions for cleavage of the C═N double bond may be suitably determined by those skilled in the art.

### 5. Method for Analyzing a Protein or Peptide

In the present invention, the fifth method is a method for analyzing a protein or peptide by mass spectrometry. This method comprises modifying, by the second method described above, a protein or peptide to be analyzed, which may be followed by subjecting the modified protein or peptide as a sample to mass spectrometry or by isolation by the fourth method and then subjecting the resulting peptide fragment as a sample to mass spectrometry.

As described in the second method, when the compound which has a group having a fluorescent chromophore is used as the nucleophilic agent, the protein or peptide modified by the method is fragmented by a suitable method, and the resulting peptide fragment mixture is separated by high performance liquid chromatography (HPLC) while monitoring with a fluorescence detector, whereby the C-terminal peptide fragment can be separated and purified. This separation procedure can be combined with a mass spectroscope to effect separation and identification of the C-terminal peptide fragment simultaneously by LC-MS. Further, the above procedure can be combined with a mass spectroscope of tandem type or equipped with an ion trap to determine an amino acid sequence of the C-terminal peptide by LC/MS/MS. As the apparatus, LC/ESI-MS and LC/ESI-MS/MS may be used. This method can also be expected to open the door to quantification method of a protein or peptide by measurement of the fluorescence intensity of a fluorescent chromophore introduced into the C-terminus.

On the other hand, when the compound having a charged group is used as the nucleophilic reagent in the second method, the protein or peptide modified by the method can be subjected directly to mass spectrometry to analyze its amino acid sequence. The protein or peptide after modification may be fragmented into a modified C-terminal peptide fragment and other peptide fragments. The method of fragmentation same as that described in the fourth method may be either a chemical method or a biochemical method. In this case too, the chemical structure derived from the C-terminal aldehyde group generated by modification (that is, a double bond or a cyclic structure formed directly with carbon derived from the aldehyde) in the present invention is resistant to the hydrolysis action of proteases. The double-bond structure and cyclic structure are as previously described in the second method. Accordingly, enzymes that can be used in fragmentation can be selected more freely than those used after immobilization of the C-terminus via an amide bond or an ester bond. That is, the selectable range of usable methods can be made broader. Specific conditions for fragmentation may be suitably determined by those skilled in the art, depending on reagents and enzymes used. The fragmented peptide fragment mixture may be used as a mass spectrometric sample directly without procedures such as isolation.

The charged group is bound to the terminus of a protein or peptide, thereby effectively facilitating formation of molecular ions and generation of fragment ions in mass analysis of the protein or peptide. Accordingly, when the peptide fragment mixture is subjected directly to mass spectrometry, a peak of the modified C-terminal peptide fragment can be distinguished from peaks of other peptide fragments, thus enabling amino acid sequencing from the C-terminus by MS and MS/MS. As the apparatus, MALDI-MS and MALDI-MS/MS may be used. As described above, the efficiency of analysis can be extremely increased by this method.

However, there is the case where the true C-terminal peptide fragment cannot be distinguished from other peptide fragments because of amino acid sequences of the peptide fragments, a charged state in the C-terminus, or the difference in the efficiency of conversion into an aldehyde group attributable to the formylation reagent used. In this case, the C-terminal peptide fragment may be purified. For this purpose, the method for isolating the C-terminal peptide fragment is preferably conducted by the fourth method for example.

Particularly in the fourth method, isolation of the C-terminal peptide fragment in a state maintaining a charged structure is effective in analysis of amino acid sequence. For isolation of the fragment in such state, a support having a cysteinyl group as the reactive group and further having a charged intervening structure is used as the nucleophilic agent. By this form, the C-terminal peptide fragment is purified and simultaneously molecular ions and fragment ions can be readily generated for analysis by mass spectrometry. Accordingly, amino acid sequencing by MS and MS/MS can be carried out easily and accurately. As the apparatus, MALDI-MS and MALDI-MS/MS may be used. As described above, the accuracy and reliability of analysis can be extremely increased by this method.

In the present invention, the fifth method comprises amino acid sequencing from the C-terminus to enable highly accurate and reliable proteome analysis. Further, this method is combined with known methods of N-terminal amino acid sequencing of a protein or peptide, thereby enabling more highly accurate and reliable proteome analysis.

### EXAMPLES

Hereinafter, the present invention is described in more detail by reference to examples where 2-hydrazino-2-imidazoline or a cysteinyl arginine resin is used as the compound having a nucleophilic group, but the present invention is not limited thereto.

### Example 1

In this example, a protein RCM-STI obtained by reduction/carboxymethylation (RCM) of all cysteine residues of soybean trypsin inhibitor (STI) was used as a sample protein. The C-terminal carboxyl group of the sample protein was converted into an aldehyde group followed by reacting the product with 2-hydrazino-2-imidazoline to generate hydrazone.

RCM-STI, 3.2 mg (0.15 μmol), was dissolved in 0.5 ml of a mixture (formylation reagent) of formic acid and trifluoroacetic anhydride in equal volumes, to give a mixed solution, and then 50 μl of a solution of pentafluorophenol 3 mg (15 μmol, 100 equivalents to STI) in formic acid was added thereto, and the mixture was heated at 60° C. for 20 minutes. After the reaction was finished, the reaction solution was concentrated under reduced pressure and evaporated to dryness. A small amount of toluene was added to the resulting residue which was then concentrated under reduced pressure and evaporated to dryness; this procedure was conducted twice. The resulting residue was dissolved in 1501 of an aqueous solution containing 4.5 mg (25 μmol) of 2-hydrazino-2-imidazoline hydrobromide salt in water, and the resulting solution was regulated with 2 μl of triethylamine to have weakly alkaline pH. After the mixture was subjected to coupling (modification) reaction at room temperature for 3 hours, 0.5 ml of 50 mM aqueous ammonium bicarbonate solution containing 0.1 mg of chymotrypsin was added to the reaction mixture followed by incubation at 37° C. for 12 hours.

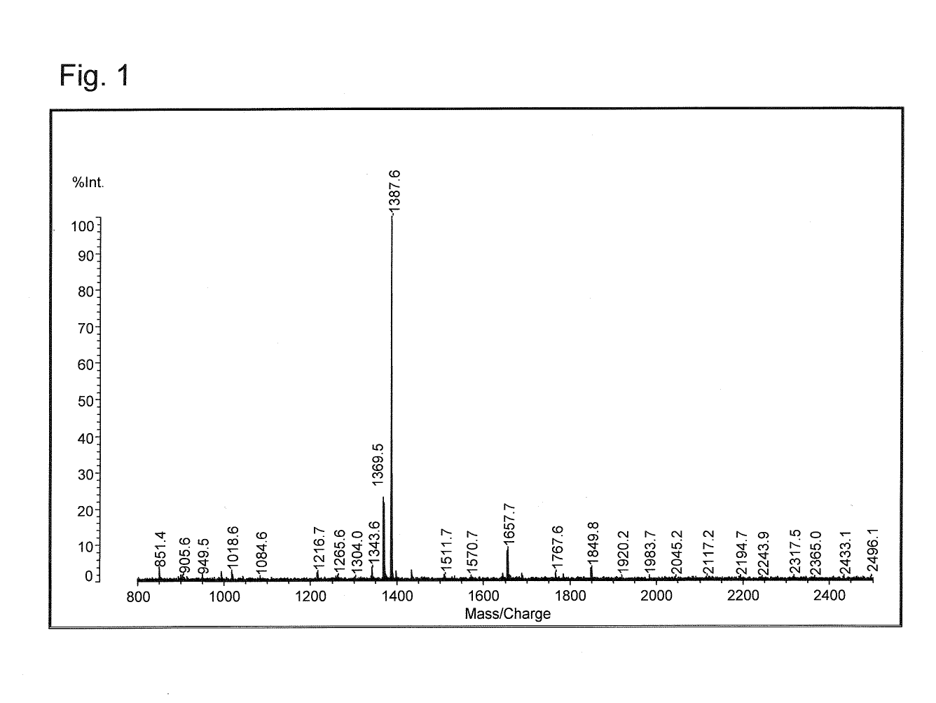

The resulting reaction product, without subjection to separation, was confirmed by MADLI-TOF MS (Axima CFR plus, manufactured by Shimadzu Corporation). The spectrum thus obtained is shown in FIG. 1. In FIG. 1, the mass/charge ratio is shown on the horizontal axis and the relative intensities of ion peaks (% int.) on the vertical axis (these apply to all spectra hereinafter). In FIG. 1, a peak of 1387.6 (m/z) is detected with the maximum intensity. From this result, it was confirmed that an ion [M+H₂+H]⁺ having 2 hydrogen atoms and a proton added to an ion (M⁺) of a peptide derivative (theoretical mass: 1384.72) obtained by conversion of the objective C-terminal peptide, that is, Gln-Phe-Gln-Lys(For)-Leu-Asp-Lys(For)-Glu-Ser(For)-Leu-H, by 2-imidazolino-2-hydrazone derivatization had been formed. Here, Leu-H indicates that the carboxyl group of C-terminal leucine is converted into an aldehyde group, and Lys (For) and Ser(For) indicate that a side chain (ε-amino group) of lysine and a side chain (β-hydroxyl group) of serine are formylated respectively.

It can be estimated that the observed MALDI peak [M+H₂+H]⁺ having a mass number higher by 3 Da than the theoretical mass is attributable to a characteristic phenomenon resulting from the reductive addition of a hydrogen molecule to a carbon-nitrogen double bond of a hydrazone group by interaction between the product and the matrix (for this phenomenon, see also Examples 3 and 5).

### Example 2

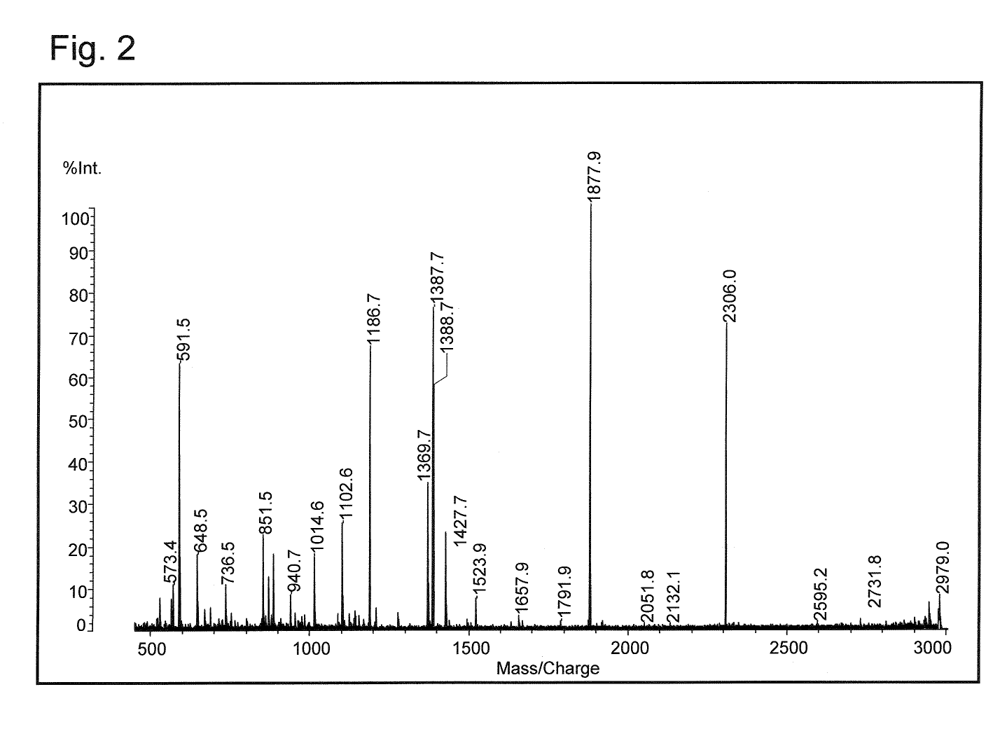

In this example, a mass spectrum was obtained in the same manner as in Example 1 except that a mixture of formic acid and acetic anhydride in equal volumes was used as the formylation reagent. The resulting mass spectrum is shown in FIG. 2. According to the analysis results in FIG. 2, the same peak of 1387.7 (m/z) was detected even when acetic anhydride was used in place of trifluoroacetic anhydride in the above example in C-terminal activation.

The results (FIG. 1) in Example 1 and the results (FIG. 2) in Example 2 show that in both the examples, a C═N bond of 2-imidazolino-2-hydrazone, derivatized from an aldehyde group of Leu that is the C-terminal amino acid of the objective C-terminal peptide, is free of hydrolysis with chymotrypsin, although there is a difference therebetween in the efficiency of conversion of the carboxyl group into an aldehyde group. That is, if the modified group is introduced via an amide bond or an ester bond without converting the carboxyl group of the Leu into an aldehyde, the amide bond or the ester bond is positioned in the terminus of the peptide chain, thus easily sterically undergoing an enzyme action, making the peptide liable to decomposition with chymotrypsin, so the objective peak cannot be detected; that is, these results show that the method of the present invention where the modified group is introduced through formation of an aldehyde group is superior to the method for introducing the modified group via an amide bond or an ester bond.

### Example 3

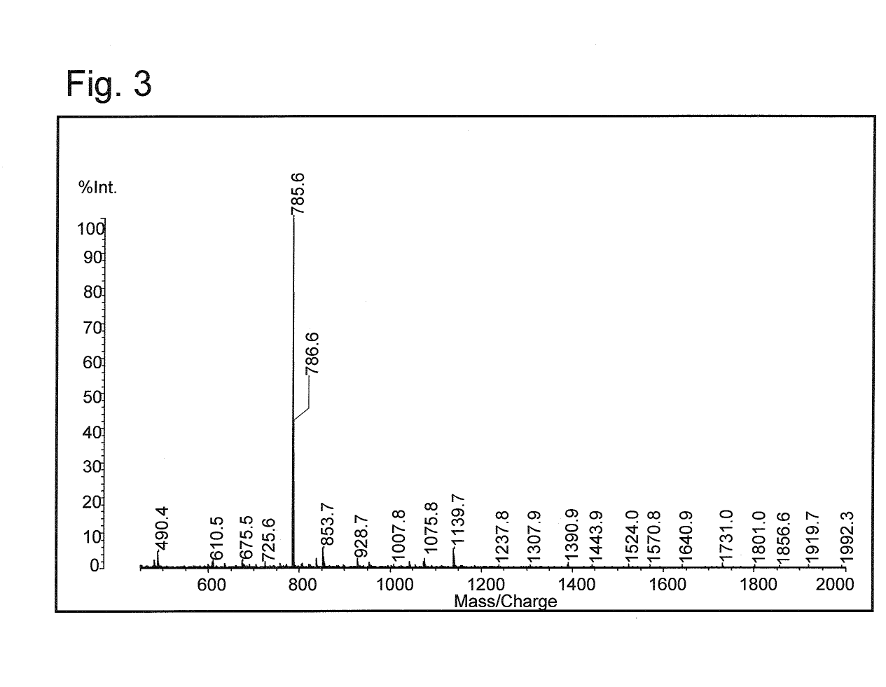

In this example, a mass spectrum was obtained in the same manner as in Example 1 except that a protein RCM-Cyt. c obtained by reduction/carboxymethylation (RCM) of all cysteine residues of horse cytochrome c (Cyt. c) was used as a sample protein. The resulting mass spectrum is shown in FIG. 3. According to the analysis results in FIG. 3, a peak of 785.6 (m/z) was detected with the maximum intensity. From this result, it was confirmed that an ion [M+H₂]⁺ having a hydrogen molecule added to an ion (M⁺) of a peptide derivative (theoretical mass: 783.42) obtained by conversion of the objective C-terminal peptide, that is, Lys(For)-Lys(For)-Ala-Thr-Asn-Glu-H (SEQ ID NO: 2), by the 2-imidazolino-2-hydrazone derivatization had been formed. Here, Glu-H indicates that the carboxyl group of C-terminal glutamic acid is converted into an aldehyde group, and Lys(For) indicates that an ε-amino group of lysine is formylated.

The observed MALDI peak [M+H₂]⁺ having a mass number higher by 2 Da than the theoretical mass, in the same manner as the peak observed in Example 1, strongly suggests that the product contains a hydrazone group having a carbon-nitrogen double bond.

### Example 4

In this example, a protein RCM-Cyt. c obtained by reduction/carboxymethylation (RCM) of all cysteine residues of horse cytochrome c (Cyt. c) was used as a sample protein. The C-terminal carboxyl group of the sample protein was converted into an aldehyde group. Using a resin having cysteinyl arginine immobilized thereon (Cys-Arg-resin) as the nucleophilic agent, the protein was immobilized onto the resin via its C-terminus, and the C-terminal peptide fragment was isolated.

A sample RCM-Cyt. c. obtained by reduction/carboxymethylation (RCM) of all cysteine residues of horse cytochrome c (Cyt. c) was used as a sample protein. The sample protein RCM-Cyt. c. was dissolved in 0.5 ml of a mixture (formylation reagent) of formic acid and trifluoroacetic anhydride in equal volumes, to give a mixed solution, and then 50 μl of a solution of 3 mg (15 μmol, 100 equivalents to STI) of pentafluorophenol in formic acid was added thereto, and the mixture was heated at 60° C. for 20 minutes. After the reaction was finished, the reaction solution was concentrated under reduced pressure and evaporated to dryness. A small amount of toluene was added to the resulting residue which was then concentrated under reduced pressure and evaporated to dryness; this procedure was conducted twice. The resulting residue was dissolved in 0.5 ml DMSO, and 0.1 g (0.22 meq/g) of Cys-Arg-resin (NovaSyn TGA, manufactured by Calbiochem & Novabiochem) was added thereto and reacted for 12 hours at room temperature.

The resin was washed with a small amount of 1% (w/w) aqueous NH₄HCO₃ solution, and then 0.5 ml of 1% (w/w) of aqueous NH₄HCO₃ solution containing 0.064 mg chymotrypsin was added thereto and incubated at 37° C. for 12 hours. After incubation, the resin was washed twice, that is, first with a small amount of 1% aqueous NH₄HCO₃ solution (w/w) and then with 70% aqueous acetonitrile solution containing 0.1% TFA (v/v/v). The washed resin was treated with an acid (i.e. subjected to a mixed solution of 82.5% trifluoroacetic acid, 5% distilled water, 5% thioanisol, 3% ethyl methyl sulfide, 2.5% ethane dithiol, and 2% thiophenol, all percentages of which are based on volume) for 4 hours, and the bound C-terminal peptide fragment was released from the resin such that the peptide contained a Cys-Arg group. The resulting solution was concentrated under reduced pressure, and the residue was analyzed by MALDI-TOF MS.

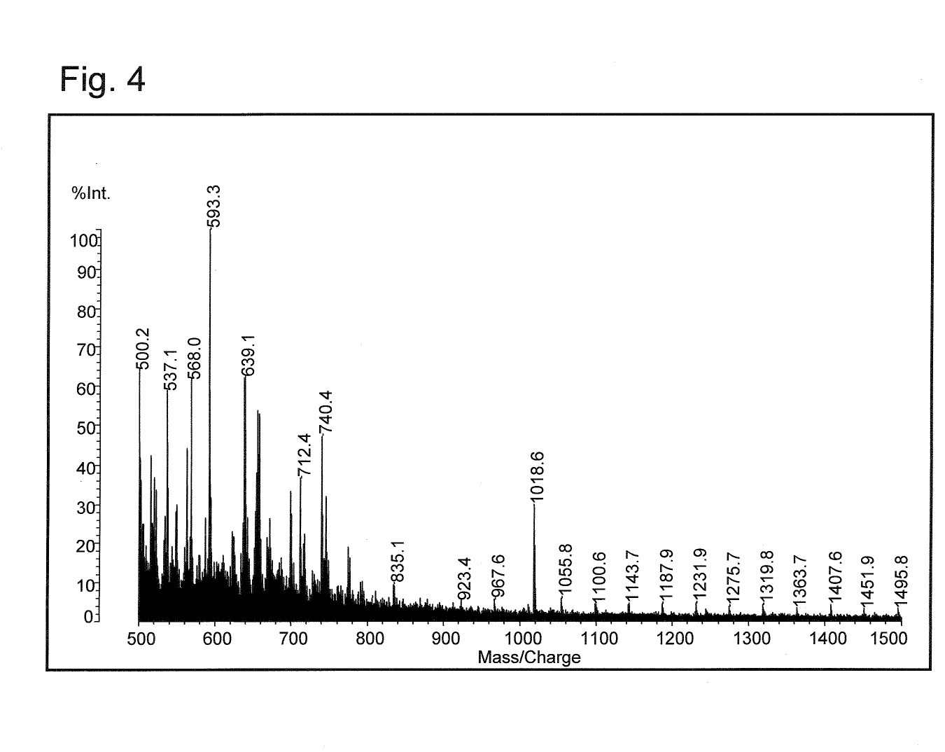

The resulting mass spectrum is shown in FIG. 4. In FIG. 4, 1018.6 (m/z) is detected in a positive-ion detection mode. Accordingly, formation of the objective product was confirmed. This objective product is in accordance with a proton-added ion 1018.52 (m/z) of the compound having a structure shown in following formula (IV). The compound of the following formula (IV), which corresponds to the C-terminal peptide fragment detected in Example 3, is a C-terminal peptide fragment that was derived from the C-terminal peptide fragment in Example 3 (Lys(For)-Lys(For)-Ala-Thr-Asn-Glu-H (SEQ ID NO: 2; Glu-H indicates that the carboxyl group of C-terminal glutamic acid is converted into an aldehyde group, and Lys(For) indicates that an ε-amino group of lysine is formylated) by further formylation of a side-chain hydroxyl group of Thr and condensation of a cysteinyl group (α-amino-β-thiol group) of the Cys-Arg resin to generate a thiazoline ring.

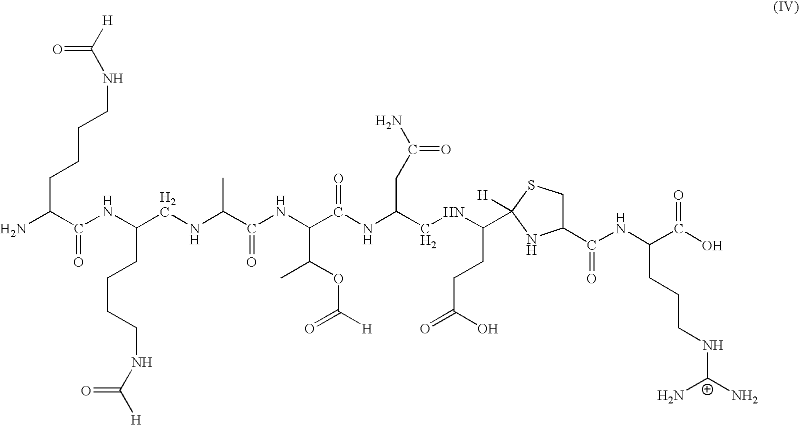

### Example 5

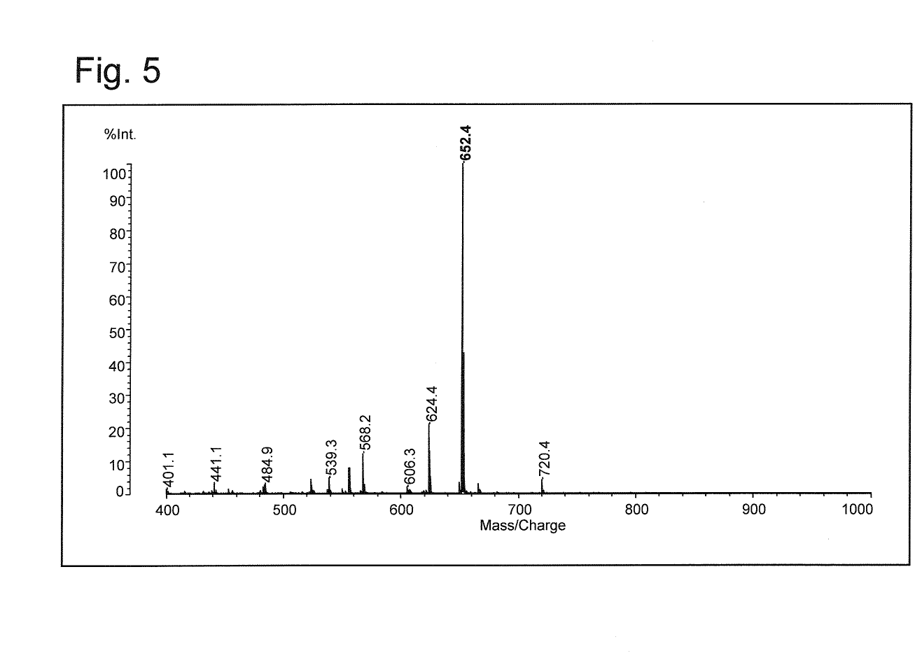

In this example, a mass spectrum was obtained in the same manner as in Example 1 except that leucine enkephalin H-Tyr-Gly-Gly-Phe-Leu-OH (SEQ ID NO: 3) was used as the sample and a chymotrypsin treatment was not conducted. The resulting mass spectrum is shown in FIG. 5.

According to the analysis results in FIG. 5, the maximum and nearly homogeneous peak is at 652.4 (m/z). This peak corresponds to a peak of an ion [M+H₂+H]⁺ having 2 hydrogen atoms and a proton added to an ion (M⁺) of a peptide derivative (theoretical mass: 649.32) obtained by conversion of the peptide For-Tyr-Gly-Gly-Phe-Leu-H by 2-imidazolino-2-hydrazone derivatization. Here, For-Tyr indicates that the amino group of N-terminal tyrosine is blocked with an N-formyl group, and Leu-H indicates that the carboxyl group of C-terminal leucine is converted into an aldehyde group. Accordingly, it was confirmed that the objective peptide derivative was formed. The observed ion [M+H₂+H]⁺ having a mass number higher by 3 Da than the theoretical mass of the above peptide derivative, in the same manner as the peaks observed in Examples 1 and 3, strongly suggests that the product contains a hydrazone group having a carbon-nitrogen double bond.

From the results in Example 1 where the ion [M+H₂⁺H]⁺ was observed, in Example 3 where the ion [M+H₂]⁺ was observed, and in Example 5 where the ion [M+H₂+H]⁺ was observed, it was confirmed that when the peptide was modified with 2-imidazolino-2-hydrazine, an MALDI peak having a mass number higher by 2 Da or 3 Da than the theoretical mass is likely to be observed rather than an MALDI peak having a mass number corresponding to the theoretical mass, depending on measurement conditions. This can be attributable to a characteristic phenomenon resulting from the reductive addition of a hydrogen molecule to a carbon-nitrogen double bond of a hydrazone group by interaction between the product and the matrix. That is, it strongly suggests that the product contains a hydrazone group having a carbon-nitrogen double bond.

In the above-described Examples, concrete 5 forms in the scope of the present invention have been shown, however, the present invention is not limited to the above-described Examples, but encompasses all of protein or peptide, formylation reagent, catalyst, and nucleophilic reagent and support having an aldehyde-reactive group. Therefore, the above-described Examples are merely exemplification in all respects, and should not be interpreted in a limitative manner. Further, any changes that belong to equivalents of claims are within the scope of the present invention.

1. A method for converting a C-terminal carboxyl group of a protein or peptide into an aldehyde group, comprising:
adding, to a protein or peptide whose C-terminal carboxyl group is to be converted into an aldehyde group,
a formylation reagent selected from a mixture of a formic acid and an acid anhydride, a mixture of a formic acid and an acid halide, and an acid anhydride having a formyl group, and
a catalyst selected from a base catalyst, an acid catalyst and a phenol catalyst,
to convert the carboxyl group into an aldehyde group.

2. The method according to claim 1, wherein the acid anhydride in the mixture of a formic acid and an acid anhydride is selected from acetic anhydride, trifluoroacetic anhydride, benzoic anhydride, o-sulfobenzoic anhydride, propionic anhydride, and pentafluoropropionic anhydride.

3. The method according to claim 1, wherein the acid anhydride having a formyl group is selected from a mixed anhydride of a formic acid and an acid other than a formic acid, and formic anhydride.

4. A method for modifying the C-terminus of a protein or peptide, comprising the steps of:
converting the C-terminal carboxyl group of a protein or peptide whose C-terminus is to be modified, into an aldehyde group by the method according to claim 1, and
reacting a nucleophilic reagent having an aldehyde-reactive group, with the aldehyde group, to modify the C-terminus.

5. The method according to claim 4, wherein the nucleophilic reagent is reacted in formic acid.

6. The method according to claim 4, wherein the aldehyde-reactive group is selected from an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—), a cysteinyl group (—COCH(NH₂)CH₂SH), and an active methylene group.

7. The method according to claim 4, wherein the aldehyde-reactive group in the nucleophilic reagent is substituted on, or bound to, an aromatic ring, an imidazoline ring and/or a fluorescent chromophore.

8. The method according to claim 4, wherein the aldehyde-reactive group in the nucleophilic reagent is substituted on, or bound to, a charged group.

9. The method according to claim 8, wherein the charged group is involved in a charged amino acid residue.

10. The method according to claim 4, wherein the nucleophilic reagent is selected from catechol, o-aminophenol, o-aminothiophenol, hydrazinobenzene, hydrazinopyrimidine, hydrazinoimidazoline, cysteine, 5-pyrazolone, and derivatives thereof.

11. The method according to claim 4, wherein the nucleophilic reagent is selected from 3,4-dihyroxybenzoic acid, 2-hydrazino-4-trifluoromethylpyrimidine, 2-hydrazino-2-imidazoline, 2-hydrazino-2-imidazoline hydrobromide salt, cysteinyl arginine, 1-methyl-3-phenyl-5-pyrazolone, and 3-methyl-1-phenyl-5-pyrazolone.

12. A method for immobilizing a protein or peptide onto a support, comprising the steps of:
converting the C-terminal carboxyl group of a protein or peptide whose C-terminus is to be immobilized, into an aldehyde group by the method according to claim 1, and
reacting a support having an aldehyde-reactive group with the aldehyde group, to immobilize the protein or peptide via the C-terminus thereof onto the support.

13. The method according to claim 12, wherein the support having an aldehyde-reactive group is reacted in formic acid.

14. The method according to claim 12, wherein the aldehyde-reactive group is selected from an amino group (—NH₂), a hydroxyl group (—OH), a thiol group (—SH), a hydrazino group (—NHNH₂), a hydroxylamino group (—NHOH), a semicarbazido group (—NHCONHNH₂), an amino group- and thiol group-substituted ethylene group (—CH(NH₂)—CH(SH)—), an amino group- and thiol group-substituted ethene group (—C(NH₂)═C(SH)—), a cysteinyl group (—COCH(NH₂)CH₂SH), and an active methylene group.

15. The method according to claim 12, wherein the aldehyde-reactive group is bound to the support via an aromatic ring, an imidazoline ring and/or a fluorescent chromophore.

16. The method according to claim 12, wherein the aldehyde-reactive group is bound via a charged group to the support.

17. The method according to claim 16, wherein the charged group is involved in a charged amino acid residue.

18. The method according to claim 12, wherein the support having an aldehyde-reactive group comprises a substance selected from catechol, o-aminophenol, o-aminothiophenol, hydrazinobenzene, hydrazinopyrimidine, hydrazinoimidazoline, cysteine, 5-pyrazolone, and derivatives thereof immobilized on the support.

19. The method according to claim 12, wherein the support having an aldehyde-reactive group comprises a substance selected from 3,4-dihyroxybenzoic acid, 2-hydrazino-4-trifluoromethylpyrimidine, 2-hydrazino-2-imidazoline, 2-hydrazino-2-imidazoline hydrobromide salt, cysteinyl arginine, 1-methyl-3-phenyl-5-pyrazolone, and 3-methyl-1-phenyl-5-pyrazolone immobilized on the support.

20. A method for isolating a C-terminal peptide fragment of a protein or peptide, comprising the step of:
immobilizing a C-terminus of a protein or peptide whose C-terminal peptide fragment is to be isolated, using a support having an aldehyde-reactive group by the method according to claim 12,
fragmenting the protein or peptide with a protease to give a C-terminal peptide fragment immobilized on the support, and other non-immobilized peptide fragments,
washing the support to remove the other non-immobilized peptide fragments, and
releasing the immobilized C-terminal peptide fragment from the support to isolate the C-terminal peptide fragment.

21. The method according to claim 20, wherein when the support having an aldehyde-reactive group has a cysteinyl group as the reactive group and the cysteinyl group is immobilized via a charged group onto the support, the C-terminal peptide fragment is isolated so as to contain the charged group in the step of isolating the C-terminal peptide fragment.

22. A method for analyzing a protein or peptide, comprising the steps of:
modifying, by the method according to claim 4, the C-terminus of a protein or peptide to be analyzed, and
subjecting the modified protein or peptide to mass spectrometry.

23. A method for analyzing a protein or peptide, comprising the steps of:
modifying, by the method according to claim 4, the C-terminus of a protein or peptide to be analyzed,
fragmenting the modified protein or peptide to give a C-terminal peptide fragment having the modified C-terminus, and other peptide fragments, and
subjecting the C-terminal peptide fragment and the other peptide fragments to mass spectrometry.

24. A method for analyzing a protein or peptide, comprising the steps of:
isolating, by the method according to claim 20, the C-terminus of a protein or peptide to be analyzed, and
subjecting the isolated C-terminal peptide fragment to mass spectrometry.


SEQUENCE LISTING (3 sequences)

SEQ ID NO: 1 - protein, 10 aa, Glycine max
QFQKLDKESL

SEQ ID NO: 2 - protein, 6 aa, Equus caballus
KKATNE

SEQ ID NO: 3 - protein, 5 aa, human
YGGFL